(19) United States
(12) Patent Application Publication
(10) Pub. No.: US 2021/0284940 A1
(43) Pub. Date: Sep. 16, 2021

(54) ENVIRONMENTAL DETECTION DEVICE

(71) Applicant: Ultimo Global Holdings, LLC, Wilmington, DE (US)

(72) Inventor: Phillip Nathan Thornberry, Carmel, IN (US)

(73) Assignee: Ultimo Global Holdings, LLC, Wilmington, DE (US)

(21) Appl. No.: 16/817,713

(22) Filed: Mar. 13, 2020

(51) Int. Cl. C12M 1/36; C12N 1/14; G05D 7/06

(52) U.S. Cl. CPC C12M 1/36; G05D 7/0676; C12N 1/14

(57) ABSTRACT

In certain aspects, the present disclosure provides unique devices and methods for detecting mold spores in a target environment. In accordance with some forms of the disclosure, such devices are configured to automatically calibrate and dynamically adjust the airflow rate through the device in response to one or more sensors.

### BACKGROUND

Environmental testing is an increasingly important aspect of evaluation of a structure. Mold or other unseen contaminants may escape the observation of an inspector. In general, mold may be detected by utilizing a spore trap sampler. Spore trap samplers function by pulling air through a sample slit, which accelerates the particles in the air stream. A small glass slide that is covered with an adhesive sampling media is positioned in the path of the air stream. The device is configured such that at optimal airflow rates mold spore particles will contact and adhere to the adhesive surface, while smaller particles move with the airflow around the glass slide. Thus the airflow rate must be controlled to ensure that as many mold spores as possible will be captured. If the flow rate is too slow, then the smaller particles, such as mold spores, will pass by and not get collected. If the flow rate is too fast, then larger particles will strike the adhesive surface of the collection media with too much force to adhere properly. Prior environmental testing devices required manual fan calibration, a process which is time consuming and prone to user error. In addition, prior devices were prone to error as environmental air movement (e.g., wind, or external fans) was commonly unaccounted for during calibration resulting in indeterminate airflow during testing. Thus, there is a need for improvement in this field.

### SUMMARY

In certain aspects, the present disclosure provides unique devices and methods for detecting mold spores in a target environment. In accordance with some forms of the disclosure, such devices are configured to automatically calibrate and dynamically adjust the airflow rate through the device in response to one or more sensors. Accordingly, in one embodiment, the present disclosure provides a device for detection of mold spores, the device comprising an air inlet, an air outlet, an airflow path extending from the air inlet to the air outlet, an airflow sensor configured to detect a flow rate of air passing through the airflow path, and a variable speed fan positioned within the airflow path, the variable speed fan configured to operate a variable speeds to maintain the flow rate of air passing through the airflow path at a designated flow rate. In some forms, the device comprises an inlet port defining the inlet opening, wherein the inlet port is configured to receive a spore trap cassette. In certain embodiments, a spore trap cassette is received on the inlet port, positioning a spore trap within the airflow path. In accordance with some embodiments, the airflow sensor comprises an airflow sensor fan having a tachometer. In some forms, the variable speed fan comprises a centrifugal fan. In some forms, the designated flow rate is about 15 liters per minute.

In accordance with some forms, the device further comprising a controller operably connected to the variable speed fan and the airflow sensor, the controller configured to adjust the speed of the variable speed fan to maintain the designated flow rate. In some forms, the controller is operably connected to one or more environmental sensors. In certain embodiments, the environmental sensors comprise one or more of the following: a temperature sensor, a pressure sensor, and/or a humidity sensor. In certain embodiments, the controller is configured to receive a signal from the environmental sensor(s) and adjust the speed of said variable speed fan in response to the signal(s).

In another embodiment, the present disclosure provides a method of maintaining a target airflow rate through a device for detection mold spores, the device having a lumen defining an airflow path. The method comprising detecting the airflow rate through the airflow path, and adjusting the speed of the fan to change the airflow rate to a designated airflow rate. In some forms, the designated flow rate is about 15 liters per minute. In some forms of practicing the disclosed methods, the airflow rate is detected by an airflow sensor positioned in the airflow path. In certain embodiments, the airflow sensor comprises an airflow sensor fan having a tachometer.

In accordance with some forms, the method further comprises the step of communicating the detected airflow rate to a controller operably connected to the variable speed fan and the airflow sensor, the controller configured to adjust the speed of said variable speed fan to maintain the designated flow rate. In certain embodiments, device further comprises one or more environmental sensors, and wherein the controller is operably connected to the environmental sensor(s), the controller configured to receive a signal from the environmental sensor(s) and adjust the speed of the variable speed fan in response to the signal(s). In certain embodiments, the environmental sensors comprise one or more of the following: a temperature sensor, a pressure sensor, and/or a humidity sensor.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a perspective view of one embodiment of an environmental sensor of the present disclosure.

FIG. 2 is a side view of one embodiment an airflow assembly of the present disclosure.

FIG. 3 is a side phantom view of one embodiment an airflow assembly of the present disclosure.

FIG. 4 is a front view of one embodiment an airflow assembly of the present disclosure.

FIG. 5 is a partial cross-sectional view of one embodiment of an airflow assembly of the present disclosure.

FIG. 6a is a front view of one embodiment of an environmental sensor of the present disclosure.

FIG. 6b is a back view of one embodiment of an environmental sensor of the present disclosure.

FIG. 6c is a left side view of one embodiment of an environmental sensor of the present disclosure.

FIG. 6d is a right side view of one embodiment of an environmental sensor of the present disclosure.

FIG. 6e is a top view of one embodiment of an environmental sensor of the present disclosure.

FIG. 6f is a bottom view of one embodiment of an environmental sensor of the present disclosure.

FIG. 7 is a side phantom view of one embodiment of an environmental sensor of the present disclosure.

FIG. 8 is a flow diagram showing one embodiment of a system for detecting a measuring mold spores.

FIG. 9a is a front perspective view of one embodiment of a spore trap cassette.

FIG. 9b is a rear perspective view of one embodiment of a spore trap cassette.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

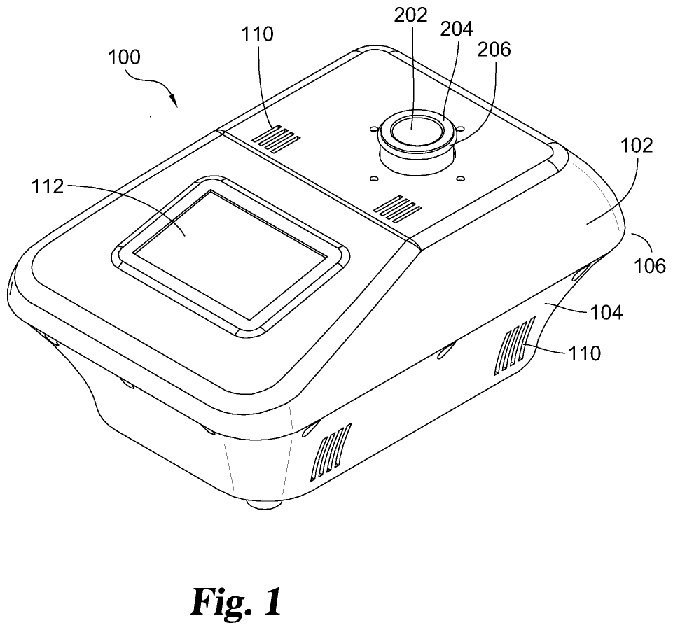

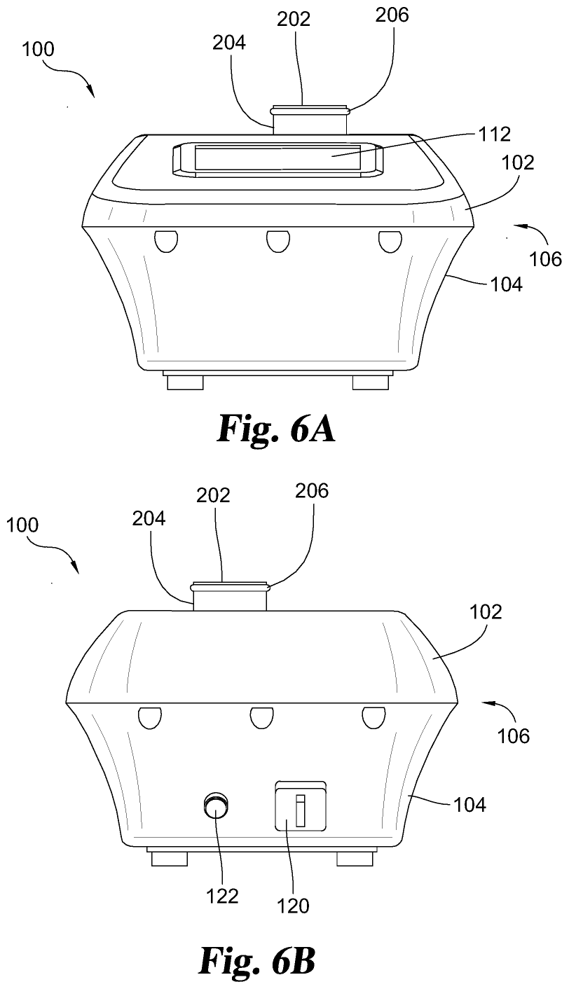

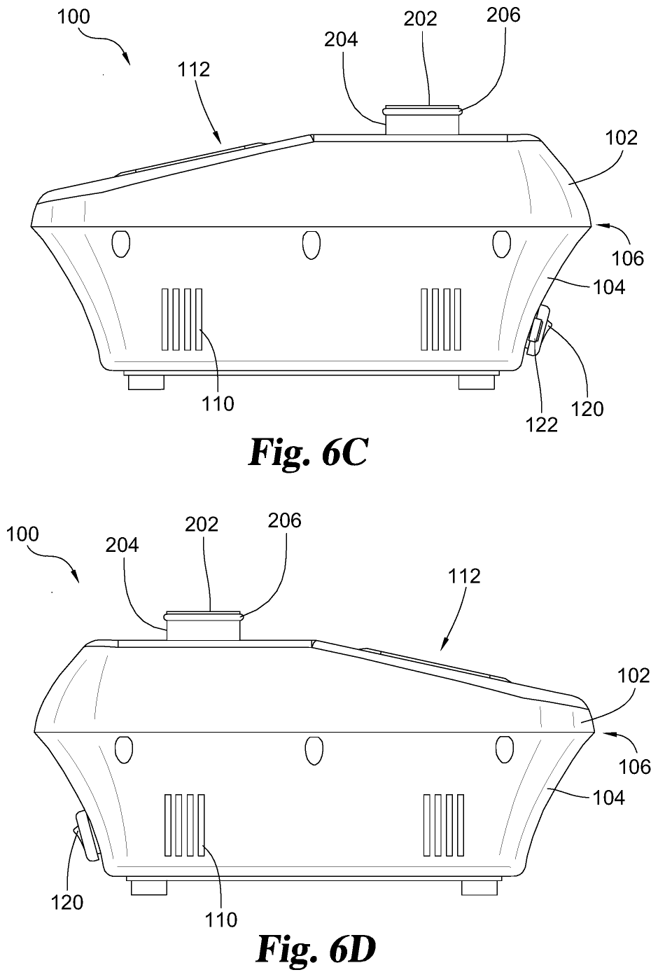

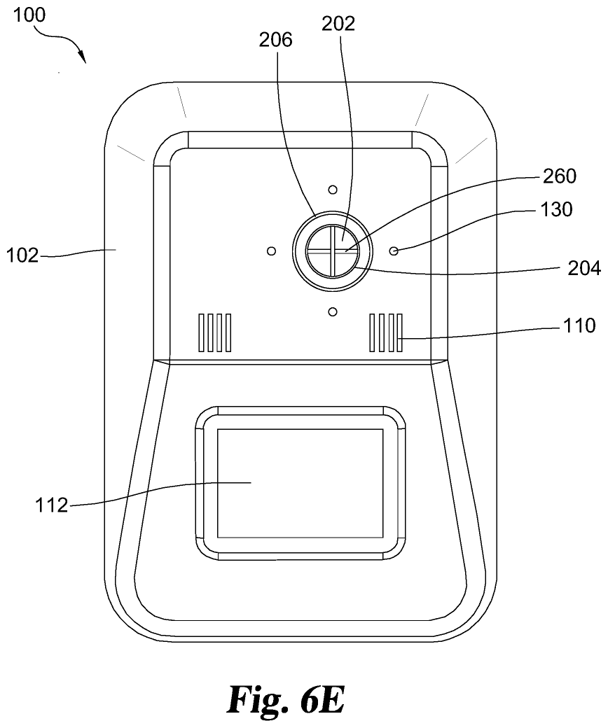

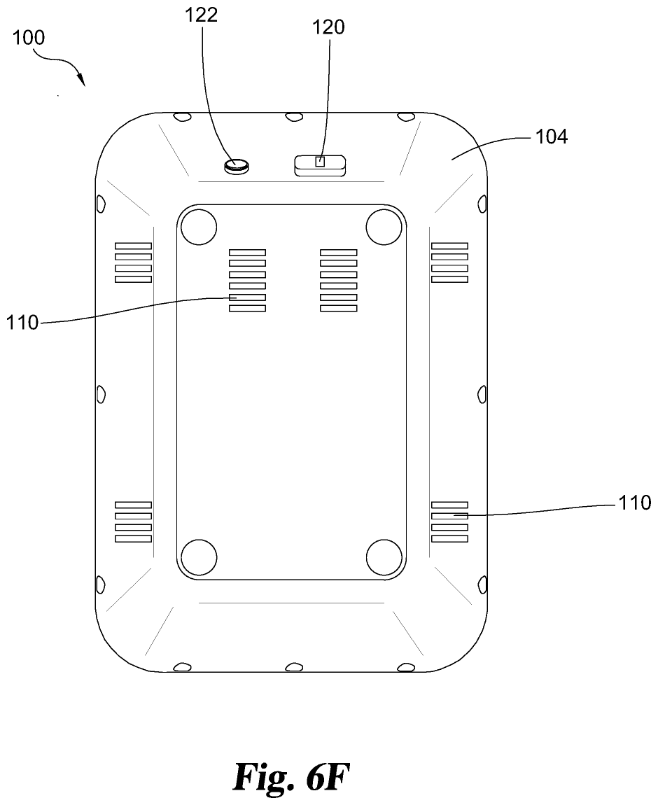

With reference to FIGS. 1, 6a, 6b, 6c, 6d, 6e, and 6f, provided are exemplary embodiments of a device for detection of mold spores 100 as disclosed herein. In the illustrated embodiments, the device comprises an upper portion 102 secured to a lower portion 104 together forming an enclosure 106. In certain embodiments, the enclosure may house one or more of the additional components described herein. In certain embodiments, the enclosure includes a plurality of ventilation openings 110, which allow air and other gases to flow into the monitoring device. In accordance with some forms, the device comprises vents configured to allow detection of an environmental parameter by an environmental sensor within the enclosure. Exemplary environmental sensors include a temperature sensor, a pressure sensor, and/or a humidity sensor. The ventilation openings may also comprise cooling vents configured to aid in cooling one or more of the components within the enclosure.

In accordance with certain embodiments, the device may include a control panel 112 configured to allow direct control of the device. In the illustrated embodiment, the control panel comprises a touch screen display.

Continuing with FIG. 1, the illustrated device further comprises an air inlet 202, having inlet port 204 configured for receiving a spore trap cassette. In certain embodiments, the port is configured to form an airtight seal with a spore trap cassette. The illustrated embodiment includes a seal 206 extending around the port to provide a seal when a spore trap cassette is positioned over the port. In some forms, the seal comprises a compliant and durable material, for example rubber.

Certain embodiments of the devices disclosed herein include a power switch 120 and/or a charging port 122. In accordance with some forms, the charging port is configured to allow charging of an internal battery. In certain embodiments, the device comprises a battery, which is configured to allow function of the device (e.g. control panel, fan and/or sensor activation and operation) without a separate power supply. In this way, the device is configured to work in remote areas in which a power supply is otherwise unavailable.

With specific reference to FIG. 6e, shown is a top down view of one embodiment of a device for detection of mold spores. In certain embodiments, the upper portion 102 of the enclosure may be adapted to secure an internal component. For example, in some forms, the upper case may comprise one or more openings 130 configured to receive a fastener or a portion of an internal component. In some forms, the device comprises upper fan support member 260.

With specific reference to FIG. 6f, shown is a bottom up view of one embodiment of a device for detection of mold spores. In some forms, the device comprises one or more vents 110 on a bottom surface of the enclosure. In accordance with some forms, such vents are aligned with one or more airflow outlet vents of an airflow system within the enclosure.

In certain embodiments, the devices disclosed herein comprise at least one airflow sensor. As used herein the term “airflow sensor” refers to a device configured to measure the amount of air per unit of time that flows through the airflow path. In certain embodiments, the airflow sensor is configured to measure the volume of air that passes through the airflow path per unit of time. For example, in a preferred embodiment the airflow sensor is configured to measure the amount of air that passes through the airflow path in liters per minute. The airflow sensor may comprise any suitable sensor, for example a Vane sensor, a hot wire sensor, a cold wire sensor, a laminar flow sensor, differential pressure flowmeters, and/or a mass flow sensor. In certain embodiments, the airflow sensor comprises an airflow sensor fan positioned within the airflow path such that the flow of air through the fluid path causes rotation of the fan. In some forms, an airflow sensor fan comprises a tachometer. As used herein, the term “tachometer” refers to a device configured to measure the rotation of the airflow sensor fan blades. In some forms, the tachometer comprises an infrared transmitter and an infrared sensor configured to sense the fan blade and determine revolutions per minute.

In some forms, the devices of the present disclosure comprise a variable speed fan. In accordance with certain embodiments, the variable speed fan is positioned to create airflow through the airflow path. In some forms, the variable speed fan comprises an axial-flow fan. IN certain preferred embodiments, the variable speed fan comprises a centrifugal fan. In some forms, the variable speed fan comprises a motor configured to run at variable speeds to increase or reduce airflow through the airflow path.

As used herein the term “Controller” generally refers to a device, using mechanical, hydraulic, pneumatic, electronic techniques, and/or a microprocessor or computer, which monitors and physically alters the operating conditions of a given dynamical system. A controller may include a processor for performing calculations to process input or output. A controller may include a memory for storing values to be processed by the processor or for storing the results of previous processing. A controller may also be configured to accept input and output from a wide array of input and output devices for receiving or sending values. Such devices include other computers, sensors, keyboards, mice, visual displays, printers, industrial equipment, and systems or machinery of all types and sizes. The communication network connected to the controller may also be connected to a wider network such as the Internet. Thus, a controller may include one or more physical processors or other computing devices or circuitry and may also include any suitable type of memory.

In certain embodiments, the present disclosure provides a device having a controller configured to monitor the flow rate of air passing through the airflow path. In some forms, the controller is configured to receive a signal from an airflow sensor. In accordance with some forms, the present disclosure provides a device having a controller configured to receive a signal from one or more sensors, for example an airflow sensor, a temperature sensor, a pressure sensor, and/or a humidity sensor. The controller may be configured to send a signal to the variable speed fan to increase or decrease the fan speed. Thus in certain embodiments, the controller is configured to receive a signal from the airflow sensor and cause the variable speed fan to adjust the fan speed towards a designated flow rate.

As discussed herein, in certain embodiments the device of the present disclosure is configured to adjust the airflow within the airflow path towards a designated flow rate. The designated flow rate may be adjusted by the user, and may be variable based on the size and/or shape of a spore trap used with the device. In accordance with some forms the device is configured to maintain airflow at a rate of about 10 liters per minute to about 20 liters per minute, preferably at a rate of about 13 liters per minute to about 17 liters per minute, even more preferably at a rate of about 14 liters per minute to about 16 liters per minute. In accordance with certain preferred embodiments, the device is configured to adjust the airflow towards a flow rate of 15 liters per minute.

In some forms, the devices of the present disclosure comprise a spore trap. In some forms, the spore trap is positioned within the airflow path. In certain embodiments, the spore trap comprises a sampling surface configured to capture mold spore particles in the air flowing through the device. In certain embodiments, the spore trap comprises an inlet funnel configured to accelerate the particles in the air stream. In certain embodiments, the air inlet funnel comprises a first opening and tapers to a second opening such that the first opening is wider than the second opening. In some forms, the sampling surface is positioned near the second opening such that the particles in the air stream are accelerated prior to contact with the sampling surface.

In accordance with some forms, the environmental detection device of the present disclosure is configured to receive and position one or more spore traps within the airflow path. Thus in some forms the environmental detection device comprises a spore trap attachment portion. The spore trap attachment portion may be configured to create an airtight seal with the spore trap. In some forms, the spore trap attachment portion comprises an o-ring or other suitable seal or gasket. In accordance with some forms the spore trap attachment portion comprises the air inlet, such that the spore trap is positioned proximally (in terms of the airflow path) to other components such as the airflow sensor or variable speed fan. Devices of the present disclosure are configured such that the spore trap is removable such that the device is reusable.

Devices of the present disclosure may include a variety of environmental sensors. For example, in some forms the devices of the present disclosure may include a temperature sensor configured to detect the ambient temperature before, during, and/or after collection of mold spores. In some forms, the devices of the present disclosure may include a pressure sensor configured to detect the ambient pressure before, during, and/or after collection of mold spores. In some forms, the devices of the present disclosure may include a humidity sensor configured to detect the ambient humidity before, during, and/or after collection of mold spores. It is also envisioned that one or more of the environmental sensors is configured to communicate the sensed environmental parameter (e.g. temperature, pressure, and/or humidity) to the controller. In some forms, the controller may adjust the fan speed in response to the sensed environmental parameter in addition to the input from the airflow sensor as changes to the temperature, pressure, and/or a humidity can affect the volume of air.

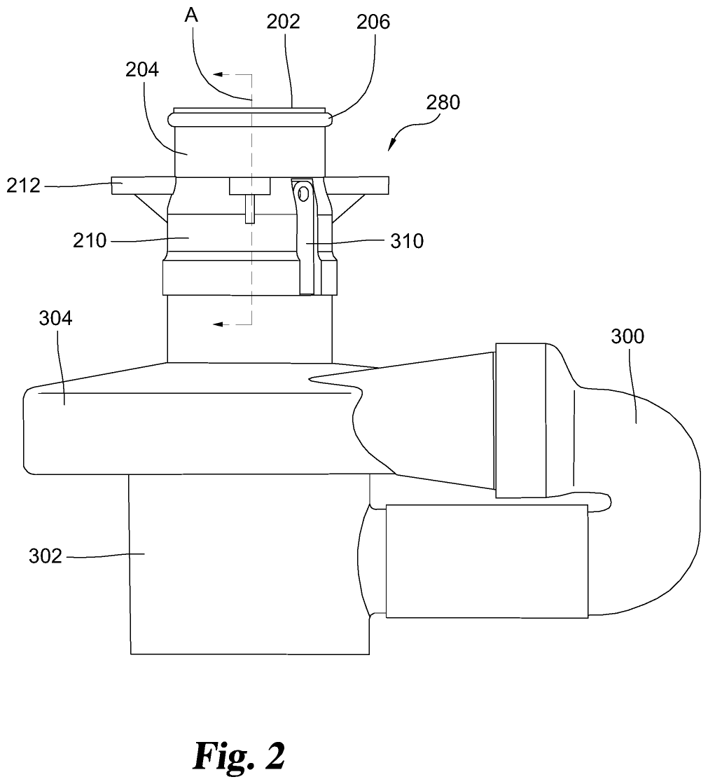

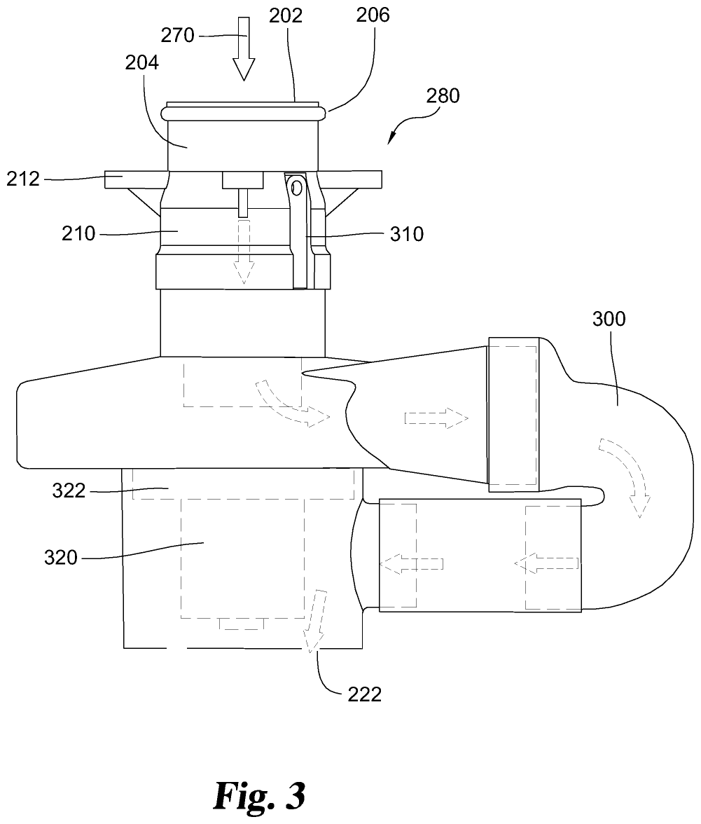

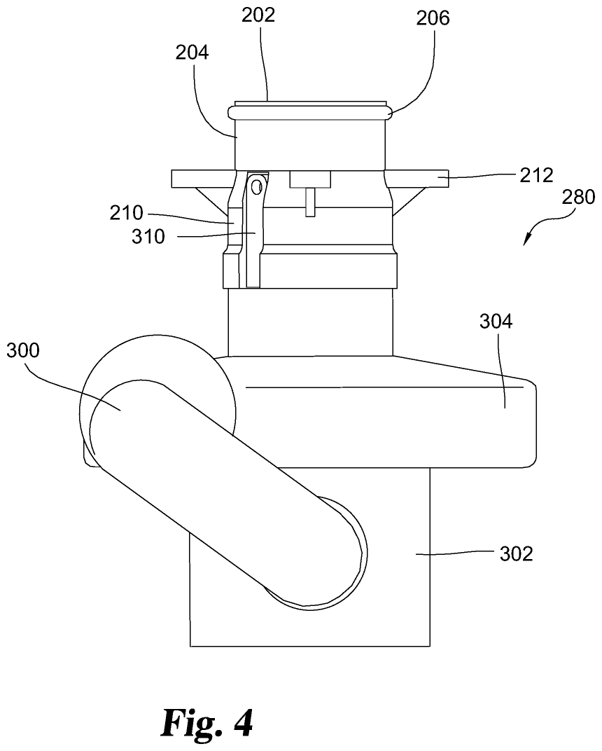

With reference to FIGS. 2, 3, and 4 shown are exemplary embodiments of an airflow assembly 280 of a device for detection of mold spores as provided herein. The enclosure may house all or part of the airflow assembly. In some forms, the airflow assembly is configured to provide a lumen between an inlet opening 202 to an outlet opening 222. Thus in some forms the device comprises an airflow path 270 within the lumen and extending from the inlet opening to the outlet opening. In certain embodiments, the device comprises an inlet port 204 at or near the inlet opening, the inlet port configured to receive a spore trap cassette. In accordance with some forms, the inlet port defines the inlet opening. In some forms, the inlet port includes seal 206 extending around a circumference of the port so as to form an airtight seal with a spore trap cassette.

In certain embodiments, the airflow assembly may comprise an airflow sensor housing 210. In some forms, the airflow sensor housing defines at least a portion of the lumen. In the illustrated embodiment the airflow sensor housing, and the airflow sensor 250 are positioned near the inlet opening, such a configuration is advantageous as it allows for airspeed measurements at a point near a spore trap cassette. It is however, within the scope of this disclosure to provide an air speed sensor and associated housing at any point along the airflow path. It is also provided within the scope of the disclosure to provide a device having multiple airspeed sensors along various points of the airflow path. As discussed herein the airflow sensor housing is configured to position an airflow sensor within the airflow path. In the illustrated embodiments, a portion of the airflow sensor, for example electrical or processing components may be stored in a recessed portion 310 of the airflow sensor housing.

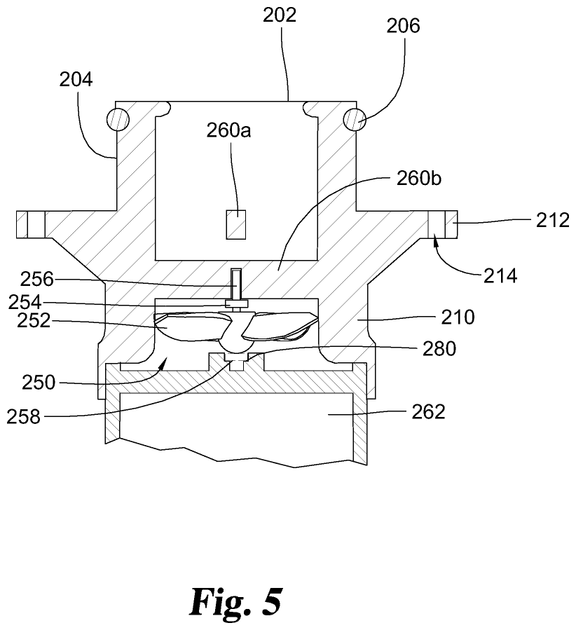

In accordance with some forms, the airflow assembly may comprise one or more support arms 212. In the illustrated embodiment, the support arms extend from the airflow sensor housing. Support arms may be configured with an opening 214 as shown in FIG. 5. In this way, the support arm is configured to pair with openings on the enclosure, see FIG. 6e, to secure the airflow assembly to the enclosure.

As discussed herein, the devices for detection of mold spores disclosed herein may comprise a variable speed fan 322. Returning to FIGS. 2, 3, 4 shown is one embodiment of fan housing 304. The fan housing is configured to define a portion of the lumen and contain some or all of the variable speed fan.

Devices of the present disclose may comprise one or more air ducts 300 configured to direct the airflow against and cool the fan motor 320. In the illustrated embodiment, air duct 300 extends from fan housing 304 to motor housing 302 and is configured to redirect the airflow such that the airflow is generally perpendicular to the fan motor surface, increasing heat exchange from the motor to the airflow.

Turning now to FIG. 5, shown is a cross-sectional view of cross section A of FIG. 2, showing a portion of the airflow assembly comprising airflow sensor housing 210 having inlet opening 202, seal 206, and inlet port 204. The airflow assembly may comprise one or more upper support members 260A and 260B. In the illustrated embodiment, the upper support members traverse the lumen, also shown in FIG. 6e, while allowing airflow through the airflow path. In some forms, the upper support members are generally perpendicular to one another. In some forms, one or more lower support members 262 are positioned below the sensor fan. As disclosed herein devices of the present disclosure may comprise an airflow sensor, the illustrated embodiment shows airspeed sensor 250 comprising sensor fan 252 and sensor shaft 256, upper bearing 254 and lower bearing 258 may also be present. In certain embodiment on or more bearing may be seated within a portion of the upper support members and/or lower support members. For example, in some forms the lower bearing is received in a bearing recess 280 on lower support member 262. In some forms, lower support member 262 comprise a beam-like structure traversing the lumen. In some forms, lower support member comprises two beams, which are generally perpendicular to one another and which intersect at the bearing recess. Thus in the illustrated embodiment, airflow through the fan causes rotation of at least the sensor fan and the sensor shaft. As disclosed herein the airflow sensor is configured to detect such rotation and interpret the speed of the airflow through the airflow path. The sensor shaft and/or bearing may be supported by one or more structural feature of the airflow assembly, for example one or more of the upper support member, and/or a lower support member.

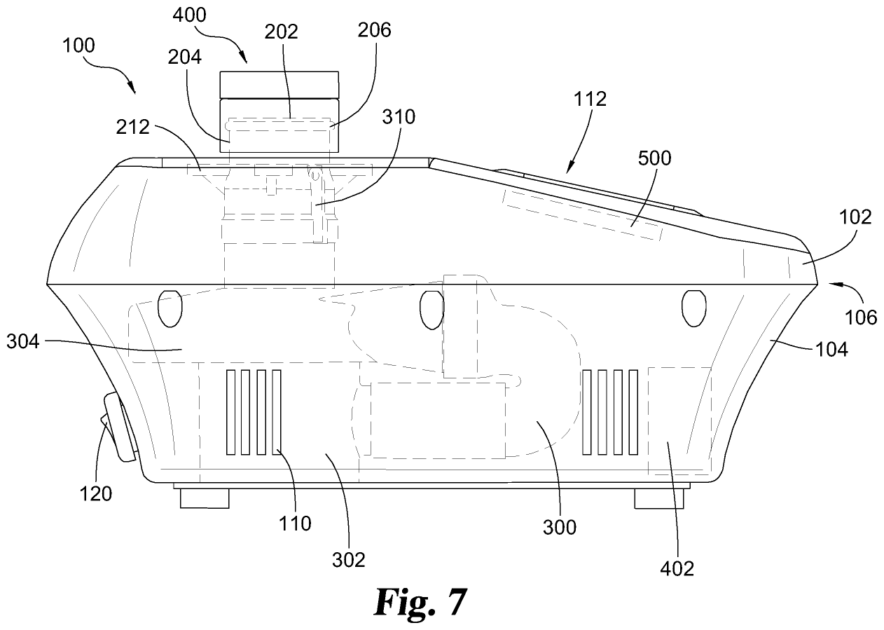

With reference to FIG. 7, shown is a side view of one embodiment of an environmental sensor of the present disclosure, showing internal components in phantom. The illustrated embodiments comprise a spore trap cassette 400 positioned on inlet port 204 such that the spore trap is positioned in the airflow path. As discussed herein, devices of the present disclosure may include a battery 402 and a controller 500 within enclosure 106.

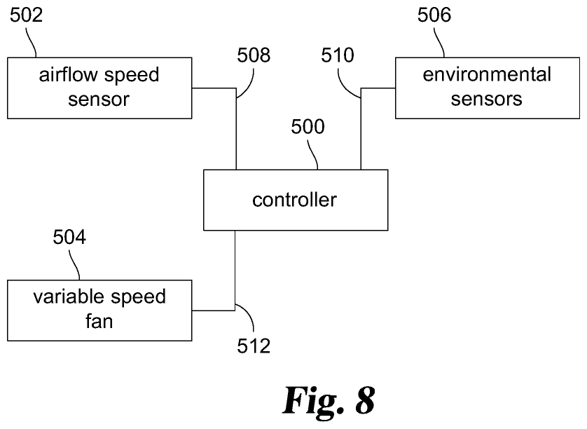

FIG. 8 is a flow diagram showing one embodiment of a system for detecting a measuring mold spores. The system comprises a controller 500, an airflow sensor 502, a variable speed fan 504, and one or more environmental sensors 506. In use, the controller communicates with the airflow sensor via transmission 508. The controller communicates with the variable speed fan to adjust the speed of the fan via transmission 512. In this way, the fan speed is adjusted to achieve a predetermined airflow at the airflow sensor. In some forms, one or more environmental sensors communicate with the controller via transmission 510. The controller may interpret the data received from the one or more environmental sensors to adjust the fan speed. Transmissions 508, 510, and/or 512 may be achieved in any suitable manner, for example a wired or wireless transmission.

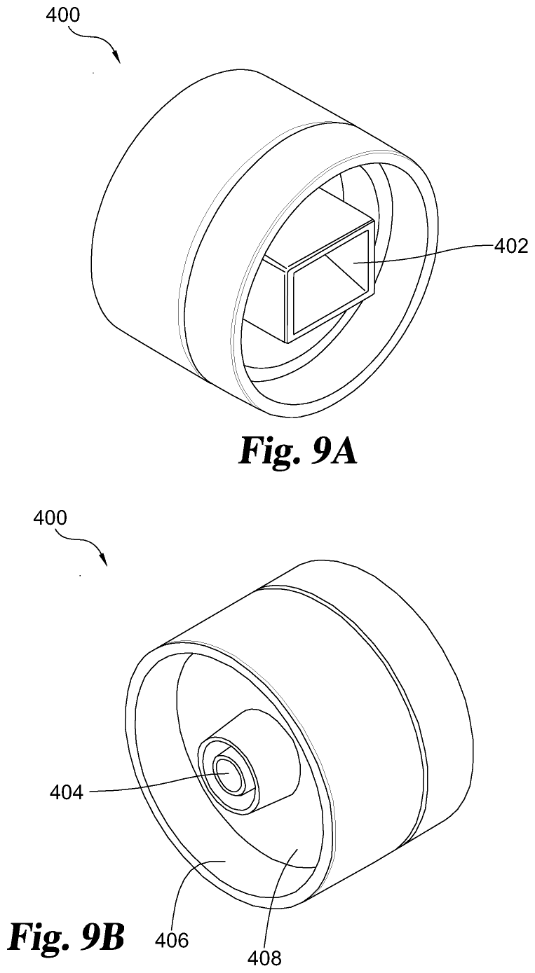

FIGS. 9A and 9B depict one embodiment of a spore trap cassette for use with the presently disclosed device. Spore trap cassette 400 comprises an air inlet 402 and an air outlet 404. In the illustrated embodiment the spore trap cassette has, a recessed portion 408 configured to receive the inlet port of the disclosed device. Spore trap cassettes for use with the preset invention are preferably configured to allow airflow through the device and against a spore trap-collecting surface while providing an airtight seal to the device.

Method

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

1. A device for detection of mold spores, the device comprising:
an air inlet;
an air outlet;
an airflow path extending from the air inlet to the air outlet;
an airflow sensor configured to detect a flow rate of air passing through the airflow path; and
a variable speed fan positioned within the airflow path, said variable speed fan configured to operate at variable speeds to maintain the flow rate of air passing through the airflow path at a designated flow rate.

2. The device of claim 1, wherein said device comprises an inlet port defining said inlet opening, and wherein said inlet port is configured to receive a spore trap cassette.

3. The device of claim 1, comprising a spore trap cassette positioned within said airflow path.

4. The device of claim 2, comprising a spore trap positioned within said airflow path, the spore trap cassette received on said inlet port.

5. The device of claim 1, wherein said airflow sensor comprises an airflow sensor fan having a tachometer.

6. The device of claim 1, wherein said variable speed fan comprises a centrifugal fan.

7. The device of claim 1, wherein said designated flow rate is about 15 liters per minute.

8. The device of claim 1, further comprising one or more environmental sensors

9. The device of claim 8, wherein said one or more environmental sensors comprises one or more of the following: a temperature sensor, a pressure sensor, and/or a humidity sensor.

10. The device of claim 1, the device further comprising a controller operably connected to said variable speed fan and said airflow sensor, said controller configured to adjust the speed of said variable speed fan to maintain the designated flow rate.

11. The device of claim 10, wherein said controller is operably connected to one or more environmental sensors, said controller configured to receive a signal from said one or more environmental sensors and adjust the speed of said variable speed fan in response to said signal.

12. The device of claim 1, wherein said variable speed fan comprises a fan motor, and wherein said fan motor is positioned within said airflow path.

13. The device of claim 1, further comprising a battery operably connected to said airflow sensor and said variable speed fan.

14. A method of maintaining a target airflow rate through a device for detection mold spores, the device having a lumen defining an airflow path, the method comprising:
detecting the airflow rate through the airflow path; and
adjusting the speed of the fan to change the airflow rate towards a designated airflow rate.

15. The method of claim 14, wherein the designated airflow rate is about 15 liters per minute.

16. The method of claim 14, wherein said detecting comprises measuring the airflow with an airflow sensor positioned in the airflow path.

17. The method of claim 14, wherein said airflow sensor comprises an airflow sensor fan having a tachometer.

18. The method of claim 14, further comprising:
communicating the detected airflow rate to a controller operably connected to said variable speed fan and said airflow sensor, said controller configured to adjust the speed of said variable speed fan to maintain the designated flow rate.

19. The method of claim 18, wherein said device further comprises one or more environmental sensors, and wherein the controller is operably connected to said one or more environmental sensors, said controller configured to receive a signal from said one or more environmental sensors and adjust the speed of said variable speed fan in response to said signal.

20. The method of claim 19, wherein said one or more environmental sensors comprises one or more of the following: a temperature sensor, a pressure sensor, and/or a humidity sensor.
